(19) United States
(12) Patent Application Publication
(10) Pub. No.: US 2024/0027253 A1
(43) Pub. Date: Jan. 25, 2024

(54) FILL LEVEL DETECTION

(71) Applicant: Cook Medical Technologies LLC, Bloomington, IN (US)

(72) Inventors: Tony Chand, Brisbane (AU); Michael Ford, Palm Beach (AU); Ravi Shrestha, Maple Grove, MN (US); Jonathan Sylvain, Lakewood, CO (US)

(73) Assignee: Cook Medical Technologies LLC, Bloomington, IN (US)

(21) Appl. No.: 18/371,756

(22) Filed: Sep. 22, 2023

Related U.S. Application Data
(63) Continuation of application No. PCT/US2022/017666, filed on Feb. 24, 2022.
(60) Provisional application No. 63/165,842, filed on Mar. 25, 2021.

(51) Int. Cl. G01F 23/263

(52) U.S. Cl. CPC G01F 23/265; F24F 2006/008

(57) ABSTRACT

A device may include a flask for holding liquid. The flask may include liquid sensors for determining a fill level of the liquid in the flask. Control circuitry may be obtain readings from the sensors and determine the fill level of the flask by applying one or single-threshold conditions for elevated fill levels and a multiple-threshold condition when determining that the fill level of the liquid is at a bottom fill level.

### CROSS-REFERENCE TO RELATED APPLICATIONS

This application is a continuation of International Application No. PCT/US2022/017666, filed Feb. 24, 2022, which claims the benefit of priority to U.S. Provisional Application No. 63/165,842, filed Mar. 25, 2021. The contents of International Application No. PCT/US2022/017666 and U.S. Provisional Application No. 63/165,842 are incorporated herein by reference in their entirety.

### TECHNICAL FIELD

This disclosure relates to a system for fill level detection in a flask.

### BACKGROUND

In various systems, the operation of a device may be dependent on the fill level of liquid in a flask. In an example, the fill level of liquid in a fuel tank may be indicative of a remaining duration of operation for vehicle. In another example, the fill level of flask in a humidification system may be indicative of the remaining duration of operation of the humidification system. In various scenarios, cessation of proper operation of devices using flasks for hosting liquids may result in catastrophic failure such as the stranding of passengers, sample dry out, or other failures. Hence, improvements in the timing of signaling for ameliorative responses to avoid failures due to low (or otherwise improper) fill levels will continue to improve device operations and user experiences.

### BRIEF DESCRIPTION OF THE DRAWINGS

FIG. 1 shows an example fill level detection device.

FIG. 2 shows a first example of fill-level detection logic is shown

FIG. 3 shows second example fill-level detection logic.

FIG. 4 shows an example capacitive fill-level detection device.

FIG. 5 shows an example gas humidification system.

### DETAILED DESCRIPTION

In various scenarios, a flask (such as a reservoir, tank, or other fluid container) may host a liquid within a device. Hosted liquid may be used in the operation of the device. In some cases, the liquid may be used during proper execution of one or more operational modes of the device. Moreover, operation of the device may deplete the liquid in the device, and/or the liquid may be depleted via processes such as evaporation, leaking, overflow, and/or other processes. Thus, the flask may be monitored to ensure the liquid is present at a fill level consistent with proper operation of the device.

In various scenarios, the flask may be opaque or have its contents otherwise obscured from view. In various scenarios, automated monitoring may be used, e.g., to ensure consistent operation. Accordingly, the flask may include liquid sensor systems to support monitoring. Liquid sensor may include capacitive sensors, optical (e.g., photoelectric) sensors, ionization sensors, and/or other sensors using transmissions (in some cases, including static fields) that extend into the flask over one or more paths between a transmitter/receiver pair. In an illustrative example, a liquid sensor may include electrodes forming a capacitor. The field in the example capacitor may extend into the flask and be affected by the liquid content therein. The capacitance between the electrodes may be used to determine the presence and/or quantity of liquid at a position within the flask.

In various scenarios, the liquid may undergo turbulence. For example, in various gas humidification applications, a gas may be forced to bubble through the liquid in the flask causing turbulence that may prevent a defined fill line from forming at the fill level of the liquid in the flask. Thus, the fill level detection scheme, e.g. used by logic executed by control circuitry within a device, may account an indefinite (or non-existent) fill line.

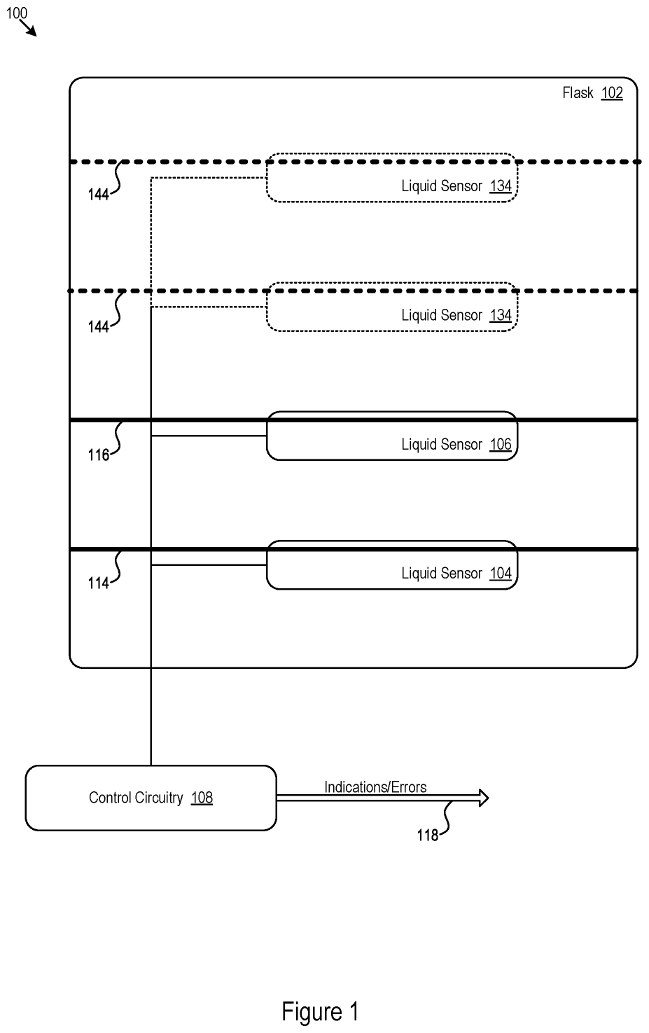

FIG. 1 shows an example fill level detection device 100. The device 100 may include a flask 102. The device 100 may include (e.g., two or more) liquid sensors 104, 106, 134 that correspond to fill levels 114, 116, 144 within the flask. The device may include control circuitry 108 that may be in communication with the liquid sensors 104, 106, 134. Using logic (e.g., example logic 200, 300), the control circuitry may generate outputs 118 (e.g., based on measured readings from the liquid sensors 104, 106, 134). The outputs may include such as indications of the fill level of the liquid within the flask 102 and/or errors such as empty flask errors, overfill errors, or other fill-level errors.

First 104 and second 106 liquid sensors may correspond to bottom and elevated sensors. A bottom sensor may correspond to a liquid sensor near or at the bottom of the flask that, for example, may correspond to a lowest fill-level for which a fill-level indication may be generated. An elevated sensor may include a sensor for which there are one or more sensors positioned below the elevated sensor. As shown in the example device 100, the second liquid sensor may correspond to a second-level fill sensor for which there is one sensor positioned below. In various implementations, bottom sensors and sensors at fill level adjacent to or otherwise near the bottom fill level may, for example, be used in the detection of empty flask conditions. Although not shown, in various implementations, first 104 and second 106 liquid sensors may include sensors located near the top of the flask and, for example, may be used in the detection of overfilled flask conditions

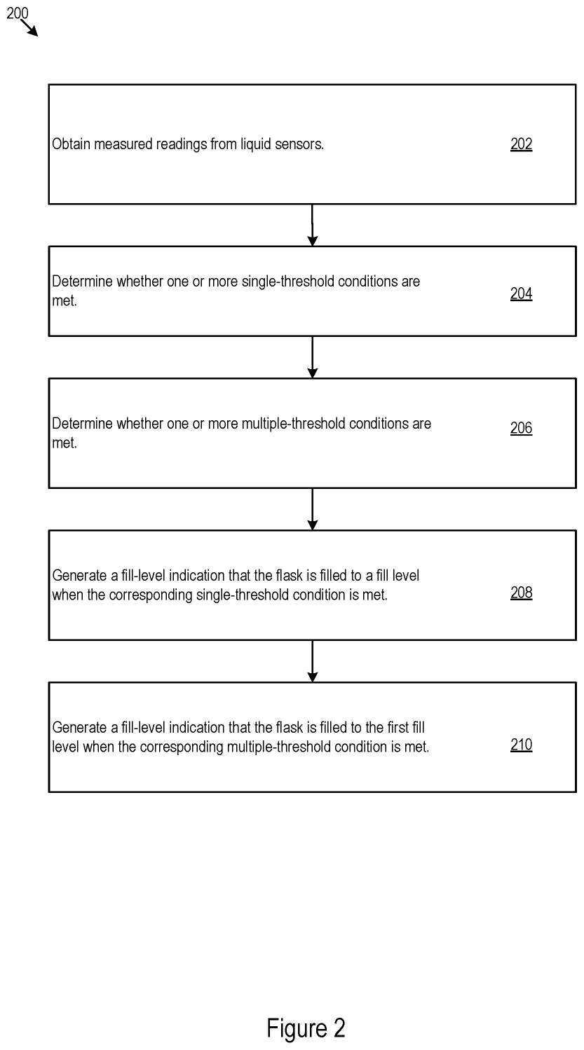

Referring now to FIG. 2 while continuing to refer to FIG. 1, a first example of fill-level detection logic FLDL 200 is shown.

The FLDL 200 may obtain measured readings from the liquid sensors 104, 106, 134 (202). Obtaining a measured reading may include receiving signal such a raw reading (e.g., an unaltered reading) from the sensor. In some cases, obtaining the measured reading may include adjusting the raw reading to correct for one or more factors.

For example, a measured reading may include an adjustment where a calibrated value (such as value from a raw reading captured while the flask is empty) may be subtracted from the current raw value (e.g., captured during normal operation).

In an example, a measured reading may include an adjustment for temperature. For example, the adjustment may be taken from a lookup table indexed for temperature and/or calculated using an algorithm that uses temperature as an input.

In various implementations, the FLDL 200 may store the measured readings in memory located on the control circuitry for later threshold comparison.

In various implementations, the FLDL 200 may poll the liquid sensors 104, 106, 134 at an interval. For example, the FLDL may poll the sensors periodically (e.g., every minute, every 20 seconds, every 10 seconds, every second) and/or at predetermined intervals.

After obtaining the measured reading from the second liquid sensor 106 and/or other liquid sensors 134, the FLDL 200 may determine whether one or more single-threshold conditions are met (204). A single-threshold condition may include a condition or set of conditions that are dependent on one or more readings from a single liquid sensor. Accordingly, a single-threshold condition for a given fill-level within a flask may be met independently of any conditions that may be present at other fill-levels. To determine whether one or more single-threshold conditions are met, the FLDL 200 may compare the measured readings from the second liquid sensor 106 and/or other liquid sensors 134 to respective fill-level thresholds for the respective fill levels of the second liquid sensor 106 and/or other liquid sensors 134. A fill-level threshold may include a predetermined value selected to indicate liquid saturation at a given fill-level. Thus, when a measured reading from a liquid sensor for a given fill-level exceeds the corresponding predetermined value for that fill-level, the corresponding single-threshold condition is met.

After obtaining the measured readings from the second liquid sensor 106 and the first liquid sensor 104, the FLDL 200 may determine whether one or more multiple-threshold conditions are met (206). A multiple-threshold condition may include a set of conditions that are dependent at least one reading from each of multiple liquid sensors each placed to obtain measurements at different fill levels. Accordingly, a measured reading from a liquid sensor for a given fill-level exceeding the corresponding fill-level threshold may be insufficient to meet a multiple-threshold condition. Rather, the multiple-threshold condition may be met when a measured reading from a liquid sensor exceeds a corresponding fill-level threshold while measured readings from one or more other liquid sensors exceed corresponding supplemental thresholds. In various implementations, a supplemental threshold for a given fill level may be lower than the corresponding fill-level threshold. Thus, such example supplemental thresholds may indicate partial liquid saturation at fill levels where the corresponding liquid sensor they are exceeded.

In various implementations, a supplemental threshold for a given fill level may be about half the corresponding fill-level threshold for that fill level. For example, the supplemental threshold may be between 40% to 60% of the corresponding fill-level threshold. For example, the supplemental threshold may be between 43% to 50% of the corresponding fill-level threshold. For example, the supplemental threshold may be 44% of the corresponding fill-level threshold. For example, the supplemental threshold may be 49% of the corresponding fill-level threshold.

In an illustrative example, a multiple-threshold condition may include a condition that measured readings from the first sensor 104 exceed a fill-level threshold and measured readings from the second sensor 106 exceed a supplemental threshold.

Accordingly, the example multiple-threshold condition may be met when liquid saturation is present at the first fill level at the same time that partial saturation is present at the second fill level. As discussed above, single-threshold conditions may be met by liquid saturation being present at the corresponding fill-level. Accordingly, multiple-threshold conditions may be more stringent than some single threshold conditions.

A multiple-threshold condition may be implemented at fill levels where spurious measured readings (e.g., unduly high (or low) readings due to turbulence or other transient effects) may lead to a failure in the operation of the system that is using the flask. For example, fill levels for which multiple-threshold conditions may be implemented may include those where such failure would occur if the actual filling of the flask is lower (or higher) than that fill level.

When the one or more of the single-threshold conditions are met, the FLDL 200 may generate one or more fill-level indications indicating that the flask is filled to the corresponding fill-level (208). When the multiple-threshold condition is met, the FLDL 200 may generate a fill-level indications indicating that the flask is filled to the first fill-level (210).

In various implementations, the FLDL 200 may send the fill-level indication corresponding to the highest fill level for which conditions (either single-threshold or multiple-threshold conditions) are met. In some cases, fill-level indication corresponding to the highest fill level may be sent regardless of whether conditions are met at lower fill levels. In some cases, fill-level indications for the lower fill levels may be omitted (e.g., not generated because the FLDL 200 may be configured to skip testing at lower fill levels when conditions for higher fill levels are met). In some cases, fill-level indications for the lower fill levels may be deleted. In some cases, the fill-level indications for the lower fill levels may be suppressed (e.g., generated and stored but not send for display and/or recordation). In some cases, the fill-level indications for the lower fill levels may be overwritten (e.g., stored at the same memory location that the FLDL 200 will later write higher fill-level indications if corresponding conditions are met).

In various implementations, if no fill-level indication is generated (e.g., because none of the conditions are met), the FLDL 200 may generate an empty flask error. For example, the FLDL 200 may generate an empty flask error when the sensors are polled and no fill level indication is generated.

The fill-level indications and empty flask errors may be sent to memory for recording and/or a display for presentation to a user. The fill levels may be presented as fill percentages (or other portion identifiers) for ease or interpretation by users. For example, a flask with four fill levels (as shown in the example device 100) may have fill levels corresponding to 25%, 50%, 75%, and 100%. Similarly, a flask with three fill levels may have fill levels corresponding to ⅓ full, ⅔ full, and full.

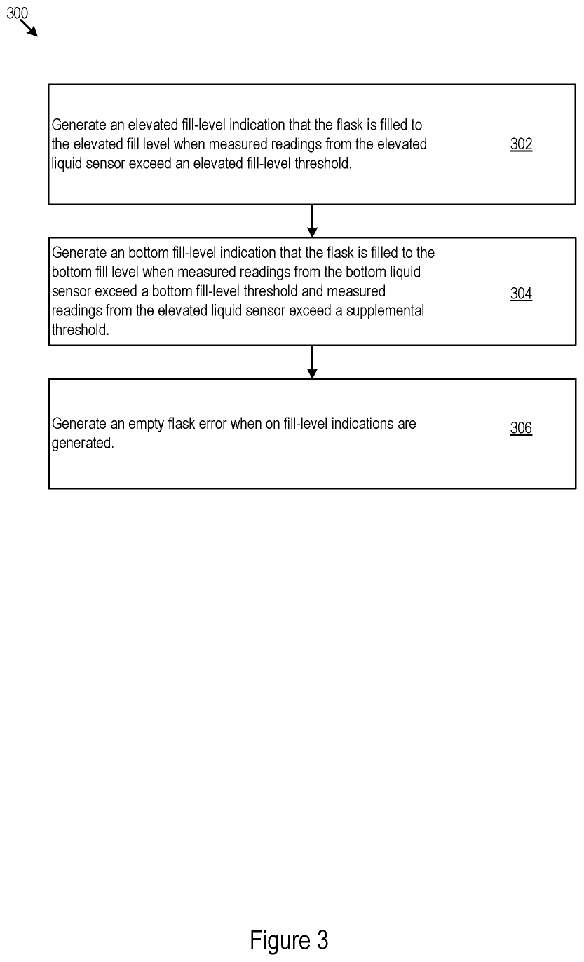

Referring now to FIG. 3 while continuing to refer to FIG. 1, second example FLDL 300 is shown. The second example FLDL 300 may also be executed (e.g., by the control circuitry 108). In various implementations, the second example FLDL 300 may also include various features of the first example FLDL 200. For example, the FLDL 300 may, in various implementations, poll sensors at intervals. For example, the FLDL 300 may, in various implementations, operate on a flask with two or more fill levels (e.g., at least a bottom fill level and one elevated fill level). The FLDL 300 may, in various implementations, operate with turbulence in the liquid within the flask. The FLDL 300 may, in various implementations, operate using measured readings that may be adjusted based on calibrated values and/or temperature-based adjustments.

In various implementations, the FLDL 300 generate an elevated fill-level indication that the flask is at the elevated fill level (e.g., the second fill level 116 or any one of the other fill levels 144) when measured readings from the elevated liquid sensor (e.g., the second liquid sensor 106 or any one of the other liquid sensors 134) exceed an elevated fill-level threshold (302).

The FLDL 300 may generate a bottom fill-level indication when measured readings from the bottom liquid sensor (e.g., the first liquid sensor 104) exceed a bottom fill-level threshold while measured readings from the elevated sensor exceed a supplementary threshold (304).

The FLDL 300 may generate an empty flask error when no fill-level indication is generated (306).

In various implementations, the FLDL 300 may suppress, delete, overwrite, or omit fill-level indications for lower levels when fill-level indications for higher levels are generated.

### Example Implementations

Various implementations have been specifically described. However, many other implementations are also possible. For example, the example implementations included below are described to be illustrative of various ones of the principles discussed above. However, the examples included below are not intended to be limiting, but rather, in some cases, specific examples to aid in the illustration of the above described techniques and architectures. The features of the following example implementations may be combined in various groupings in accord with the techniques and architectures describe above.

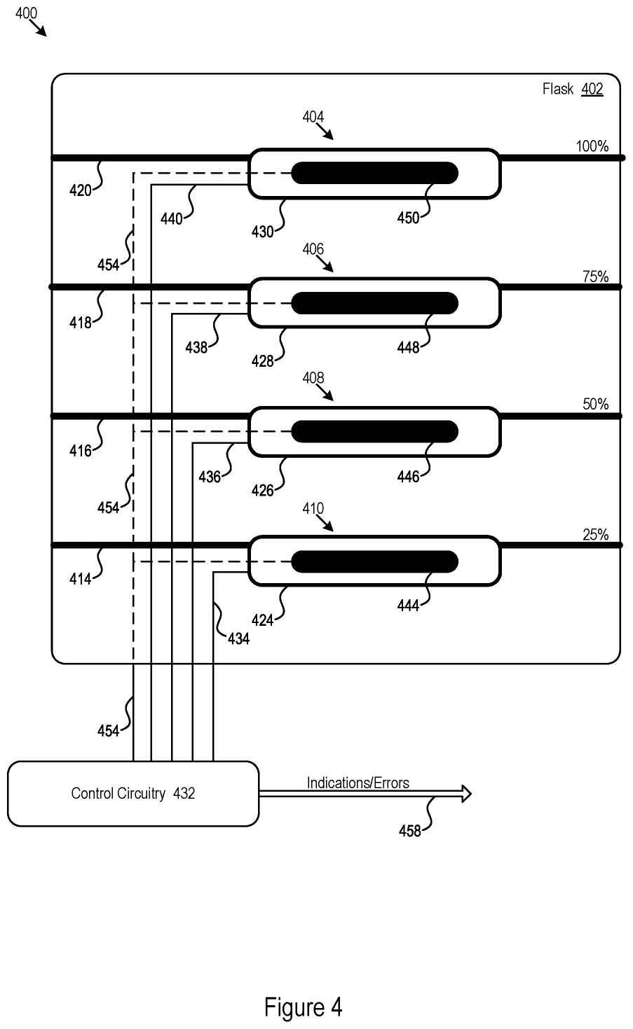

Referring to FIG. 4, an example capacitive fill-level detection device 400 is shown. The example device 400 may include a flask 402. The device 400 may include four capacitive liquid sensors 404, 406, 408, 410 that correspond to fill levels 414, 416, 418, 420 within the flask. The four capacitive sensors may each include an outer electrode 424, 426, 428, and 430, which may be individually communicatively coupled to the control circuitry 432 via lines 434, 436, 438, and 440. The four capacitive sensors may each include an inner electrode 444, 446, 448, and 450, which may be on the same plane as the outer electrode (on the outer wall of the flask) and communicatively coupled to the control circuitry 432 as a group via line 454. Using logic (e.g., example logic 300), the control circuitry 432 may generate outputs 458 (e.g., based on measured readings from the capacitive liquid sensors 404, 406, 408, 410). The fill levels 414, 416, 418, 420 correspond to fill levels of 25%, 50%, 75%, and 100% respectively.

The inner electrodes may generate pulses (or other transient signals). The amount of energy that reaches the respective outer electrode from the pulse may depend on the liquid content at the corresponding fill level within the flask. The electrical coupling between the inner and outer electrodes is increased when the liquid content increases at the corresponding fill level within the flask which changes the capacitance of the sensor and increases the amount of energy transferred between the electrodes.

In various implementations, the capacitive liquid sensors 404, 406, 408, 410 may be based on commercially available touchscreen capacitive sensors.

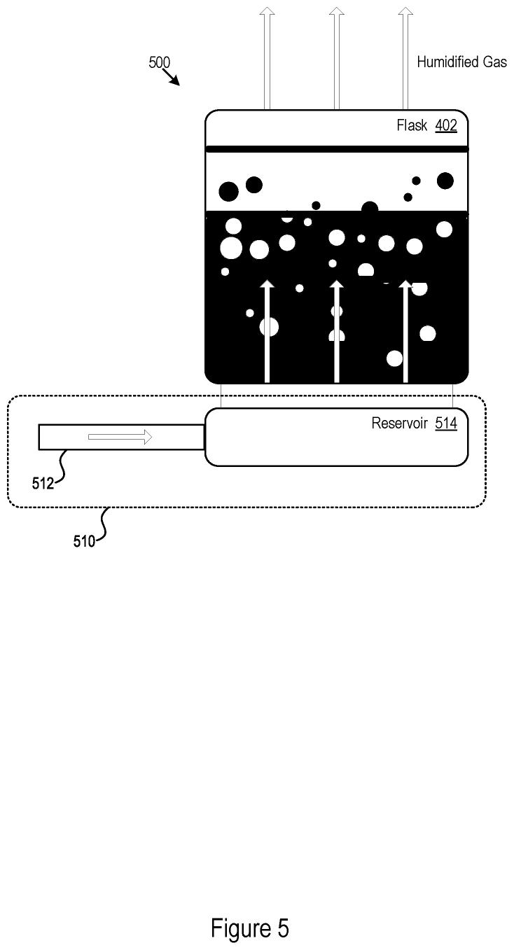

FIG. 5 shows an example gas humidification system (GHS) 500 that may implement the example capacitive fill-level detection device 400. The example GHS 500 may include the fill-level detection device 400 (including the flask 402). The example GHS 500 may further include a gas injection channel 510. The gas injection channel may be coupled to the flask 402 to force a bubbling liquid-gas mixture upward into the flask 402.

The gas injection channel 510 may include a gas injection line 512 and a reservoir 514 coupled to the gas injection line 512. The reservoir 514 may be coupled to the flask 402 from the bottom. The reservoir 514 may hold a liquid (such as water) that may be force upward into the flask 402 when gas is sent into the reservoir 514 via the gas injection line 512.

The GHS 500 may humidify the injected gas via exposure of the gas to the liquid as it bubbles upward through the flask 402.

The GHS 500 may be used in various gas humidification systems. For example, the GHS 500 may be used to humidify gas used in an incubation system, such as embryonic incubators used in assistive reproductive technology (ART) systems.

In various applications, multiple gas humidification systems (such as the example GHS 500) may be implemented to operate in parallel within a unified device. The multiple gas humidification systems may operate identically or may differ from one another in flask structure and/or operative parameters (such as fill-level thresholds, temperature adjustments, supplemental thresholds, and/or other operative parameters).

The methods, devices, processing, and logic described above may be implemented in many different ways and in many different combinations of hardware and software. For example, all or parts of the implementations may be circuitry that includes an instruction processor, such as a Central Processing Unit (CPU), microcontroller, or a microprocessor; an Application Specific Integrated Circuit (ASIC), Programmable Logic Device (PLD), or Field Programmable Gate Array (FPGA); or circuitry that includes discrete logic or other circuit components, including analog circuit components, digital circuit components or both; or any combination thereof. The circuitry may include discrete interconnected hardware components and/or may be combined on a single integrated circuit die, distributed among multiple integrated circuit dies, or implemented in a Multiple Chip Module (MCM) of multiple integrated circuit dies in a common package, as examples.

The circuitry may further include or access instructions for execution by the circuitry. The instructions may be embodied as a signal and/or data stream and/or may be stored in a tangible storage medium that is other than a transitory signal, such as a flash memory, a Random Access Memory (RAM), a Read Only Memory (ROM), an Erasable Programmable Read Only Memory (EPROM); or on a magnetic or optical disc, such as a Compact Disc Read Only Memory (CDROM), Hard Disk Drive (HDD), or other magnetic or optical disk; or in or on another machine-readable medium. A product, such as a computer program product, may particularly include a storage medium and instructions stored in or on the medium, and the instructions when executed by the circuitry in a device may cause the device to implement any of the processing described above or illustrated in the drawings.

The implementations may be distributed as circuitry, e.g., hardware, and/or a combination of hardware and software among multiple system components, such as among multiple processors and memories, optionally including multiple distributed processing systems. Parameters, databases, and other data structures may be separately stored and managed, may be incorporated into a single memory or database, may be logically and physically organized in many different ways, and may be implemented in many different ways, including as data structures such as linked lists, hash tables, arrays, records, objects, or implicit storage mechanisms. Programs may be parts (e.g., subroutines) of a single program, separate programs, distributed across several memories and processors, or implemented in many different ways, such as in a library, such as a shared library (e.g., a Dynamic Link Library (DLL)). The DLL, for example, may store instructions that perform any of the processing described above or illustrated in the drawings, when executed by the circuitry.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A device including:
a flask for a liquid;
a first liquid sensor placed to measure a first amount of liquid present at a first fill level within the flask;
a second liquid sensor placed to measure a second amount of liquid present at a second fill level within the flask; and
control circuitry in operative communication with the first and second liquid sensors, the control circuitry configured to:
obtain a first measured reading from the first sensor, the first measured reading indicative of the first amount;
obtain a second measured reading from the second sensor, the second measured reading indicative of the second amount;
determine that one or more single-threshold conditions are met at a time that the second measured reading exceeds a second fill-level threshold;
determine that a multiple-threshold condition is met at a time that:
the first measured reading exceeds a first fill-level threshold; and
the second measured reading exceeds a supplemental threshold,
the supplemental threshold different than the second fill-level threshold;
generate an elevated fill-level indication that the flask is filled at least to the second fill level at a time that the one or more single-threshold conditions are met; and
generate a first fill-level indication that the flask if filled to the first fill level at a time that the multiple-threshold condition is met.

2. The device of claim 1, wherein the flask includes:
a third fill level; and
a fourth fill level,
wherein the control circuitry is configured to generate an elevated fill-level indication by generating an indication that the flask is filled to the second fill level, the third fill level, or the fourth fill level, wherein the first, second, third, and fourth fill levels correspond to 25%, 50%, 75%, and 100% fill-level indications, respectively.

3. The device of claim 1, wherein the device further includes:
a third liquid senor placed to measure a third amount of liquid present at a third fill level within the flask, wherein the control circuitry is further configured to:
determine that the one or more single-threshold conditions are met at a time that the third measured reading exceeds a third fill-level threshold; and
generate the elevated fill-level indication that the flask is filled at least to the third fill level at a time that:
the one or more single-threshold conditions are met; and
the third measured reading exceeds a third fill-level threshold.

4. The device of claim 3, where the device further includes:
a fourth liquid senor placed to measure a fourth amount of liquid present at a fourth fill level within the flask, wherein the control circuitry is further configured to:
determine that the one or more single-threshold conditions are met at a time that the fourth measured reading exceeds a fourth fill-level threshold; and
generate the elevated fill-level indication that the flask is filled at least to the fourth fill level at a time that:
the one or more single-threshold conditions are met; and
the fourth measured reading exceeds a fourth fill-level threshold.

5. The device of claim 1, wherein the control circuitry is further configured to generate an empty flask error at a time that no fill-level indication is generated.

6. The device of claim 1, wherein the control circuitry is further configured to generate a highest fill-level indication for a highest fill level for which a single-threshold condition, a multiple-threshold condition or both are met regardless of whether conditions are met for one or more fill levels below the highest fill level.

7. The device of claim 6, wherein the control circuitry is further configured to suppress a fill-level indication corresponding to any of the fill levels below the highest fill level.

8. The device of claim 1, where the flask is configured to hold the liquid in a turbulent state.

9. The device of claim 8, where the turbulent state includes bubbling due to gas injection for humidification.

10. The device of claim 1, where the supplemental threshold is set to about half of the second fill-level threshold, wherein:
the supplemental threshold is set between 40% to 60% of the second fill-level threshold;
the supplemental threshold is set between 43% to 50% of the second fill-level threshold;
the supplemental threshold is set to 44% of the second fill-level threshold; or
the supplemental threshold is set to 49% of the second fill-level threshold.

11. The device of claim 1, wherein the control circuitry is further configured to obtain the first measured reading by subtracting a calibrated value from a raw reading from the first liquid sensor.

12. The device of claim 1, wherein the control circuitry is further configured to obtain the first measured reading by applying a temperature-dependent adjustment to a raw reading from the first liquid sensor.

13. The device of claim 1, where the first and second liquid sensors are placed to support:
generation of error messages by the control circuitry where the flask filled below a minimum operational tolerance; or
generation of error messages by the control circuitry where the flask filled above a maximum operational tolerance.

14. The device of claim 1, where the control circuitry is further configured to:
poll the liquid sensors at an interval; and
generate an empty flask error at a time that the poll of the liquid sensors does not result in generation of a fill-level indication,
wherein the interval includes at least one of a predetermined interval or a periodic interval.

15. A device including:
a flask;
a bottom liquid sensor corresponding to a bottom fill level of the flask;
an elevated liquid sensor corresponding to an elevated fill level of the flask, the elevated fill level above the bottom fill level; and
control circuitry in operative communication with the first and second liquid sensors, the control circuitry configured to:
generate an elevated fill-level indication that the flask is at the elevated fill level at a time that a measured reading from the elevated liquid sensor exceeds an elevated fill-level threshold;
generate a bottom fill-level indication that the flask is at the bottom fill level at a time that:
a measured reading from the bottom liquid sensor exceeds a bottom fill-level threshold; and
the measured reading from the elevated liquid sensor exceeds a supplementary threshold; and
generate an empty flask error at a time that no fill-level indication is generated.

16. The device of claim 15, wherein the bottom fill level includes a lowest fill level for which the control circuitry is configured to generate a fill-level indication.

17. The device of claim 15, wherein the elevated fill level includes a fill level adjacent to the bottom fill level.

18. The device of claim 15, wherein the device includes one or more fill levels above the elevated fill level.

19. The device of claim 15, wherein the control circuitry is further configured to suppress the bottom fill-level indication at a time that:
the measured reading from the bottom liquid sensor exceeds the bottom fill-level threshold; and
the measured reading from the elevated liquid sensor exceeds the elevated fill-level threshold.

20. A device including:
a flask;
a gas injection channel coupled to the flask to force a bubbling liquid-gas mixture upward into the flask;
a bottom capacitive sensor placed to measure a liquid content of the bubbling liquid-gas mixture at a bottom fill level of the flask;
a second fill-level capacitive sensor placed to measure a liquid content of the bubbling liquid-gas mixture at an second fill level of the flask, the second fill level above the bottom fill level and adjacent to the bottom fill level; and
control circuitry in operative communication with the bottom and second fill-level liquid sensors, the control circuitry configured to:
generate a second fill-level indication that the flask is at the second fill level at a time that the liquid content of the bubbling liquid-gas mixture at the second fill level exceeds a second fill-level threshold;
generate a bottom fill-level indication that the flask is at the bottom fill level at a time that:
the liquid content of the bubbling liquid-gas mixture at the bottom fill level exceeds a bottom fill-level threshold; and
the liquid content of the bubbling liquid-gas mixture at the second fill level exceeds a supplementary threshold; and
generate an empty flask error at a time that no fill-level indication is generated.
